(19) United States
(12) Patent Application Publication
(10) Pub. No.: US 2004/0039441 A1
(43) Pub. Date: Feb. 26, 2004

(54) Drug eluting implantable medical device

(76) Inventors: Stephen Maxwell Rowland, Miami, FL (US); Ike Juman, Davie, FL (US); Robert John Cottone JR., Davie, FL (US); David Lawrence Camp JR., Fort Lauderdale, FL (US)

Correspondence Address: WHITE & CASE LLP, PATENT DEPARTMENT, 1155 AVENUE OF THE AMERICAS, NEW YORK, NY, 10036, US

(21) Appl. No.: 10/442,669

(22) Filed: May 20, 2003

Related U.S. Application Data
(60) Provisional application No. 60/382,095, filed on May 20, 2002.

(51) Int. Cl. A61F 2/06

(52) U.S. Cl. 623/1.42; 427/2.1

(57) ABSTRACT

A drug eluting medical device is provided for implanting into vessels or luminal structures within the body of a patient. The coated medical device, such as a stent, vascular, or synthetic graft comprises a coating consisting of a controlled-release matrix of a bioabsorbable, biocompatible, bioerodible, biodegradable, nontoxic material, such as a Poly(DL-Lactide-co-Glycolide) polymer, and at least one pharmaceutical substance, or bioactive agent incorporated within the matrix or layered within layers of matrix. In particular, the drug eluting medical device when implanted into a patient, delivers the drugs or bioactive agents within the matrix to adjacent tissues in a controlled and desired rate depending on the drug and site of implantation.

This application claims priority to U.S. Provisional Patent Application Serial No. 60/382,095, filed on May 20, 2002.

### FIELD OF INVENTION

The invention relates to a medical device implanted in vessels or luminal structures within the body. More particularly, the present invention relates to stents and synthetic grafts which are coated with a controlled-release matrix comprising a medicinal substance for direct delivery to the surrounding tissues. In particular, the drug-coated stents are for use, for example, in balloon angioplasty procedures for preventing restenosis.

### BACKGROUND OF INVENTION

Atherosclerosis is one of the leading causes of death and disability in the world. Atherosclerosis involves the deposition of fatty plaques on the luminal surface of arteries. The deposition of fatty plaques on the luminal surface of the artery causes narrowing of the cross-sectional area of the artery. Ultimately, this deposition blocks blood flow distal to the lesion causing ischemic damage to the tissues supplied by the artery.

Coronary arteries supply the heart with blood. Coronary artery atherosclerosis disease (CAD) is the most common, serious, chronic, life-threatening illness in the United States, affecting more than 11 million persons. The social and economic costs of coronary atherosclerosis vastly exceed that of most other diseases. Narrowing of the coronary artery lumen causes destruction of heart muscle resulting first in angina, followed by myocardial infarction and finally death. There are over 1.5 million myocardial infarctions in the United States each year. Six hundred thousand (or 40%) of those patients suffer an acute myocardial infarction and more than three hundred thousand of those patients die before reaching the hospital. (Harrison's Principles of Internal Medicine, 14th Edition, 1998).

CAD can be treated using percutaneous transluminal coronary balloon angioplasty (PTCA). More than 400,000 PTCA procedures are performed each year in the United States. In PTCA, a balloon catheter is inserted into a peripheral artery and threaded through the arterial system into the blocked coronary artery. The balloon is then inflated, the artery stretched, and the obstructing fatty plaque flattened, thereby increasing the cross-sectional flow of blood through the affected artery. The therapy, however, does not usually result in a permanent opening of the affected coronary artery. As many as 50% of the patients who are treated by PTCA require a repeat procedure within six months to correct a re-narrowing of the coronary artery. Medically, this re-narrowing of the artery after treatment by PTCA is called restenosis. Acutely, restenosis involves recoil and shrinkage of the vessel. Subsequently, recoil and shrinkage of the vessel are followed by proliferation of medial smooth muscle cells in response to injury of the artery from PTCA. In part, proliferation of smooth muscle cells is mediated by release of various inflammatory factors from the injured area including thromboxane A2, platelet derived growth factor (PDGF) and fibroblast growth factor (FGF). A number of different techniques have been used to overcome the problem of restenosis, including treatment of patients with various pharmacological agents or mechanically holding the artery open with a stent. (Harrison's Principles of Internal Medicine, 14th Edition, 1998).

Of the various procedures used to overcome restenosis, stents have proven to be the most effective. Stents are metal scaffolds that are positioned in the diseased vessel segment to create a normal vessel lumen. Placement of the stent in the affected arterial segment prevents recoil and subsequent closing of the artery. Stents can also prevent local dissection of the artery along the medial layer of the artery. By maintaining a larger lumen than that created using PTCA alone, stents reduce restenosis by as much as 30%. Despite their success, stents have not eliminated restenosis entirely. (Suryapranata et al. 1998. Randomized comparison of coronary stenting with balloon angioplasty in selected patients with acute myocardial infarction. Circulation 97:2502-2502).

Narrowing of the arteries can occur in vessels other than the coronary arteries, including the aortoiliac, infrainguinal, distal profunda femoris, distal popliteal, tibial, subclavian and mesenteric arteries. The prevalence of peripheral artery atherosclerosis disease (PAD) depends on the particular anatomic site affected as well as the criteria used for diagnosis of the occlusion. Traditionally, physicians have used the test of intermittent claudication to determine whether PAD is present. However, this measure may vastly underestimate the actual incidence of the disease in the population. Rates of PAD appear to vary with age, with an increasing incidence of PAD in older individuals. Data from the National Hospital Discharge Survey estimate that every year, 55,000 men and 44,000 women had a first-listed diagnosis of chronic PAD and 60,000 men and 50,000 women had a first-listed diagnosis of acute PAD. Ninety-one percent of the acute PAD cases involved the lower extremity. The prevalence of comorbid CAD in patients with PAD can exceed 50%. In addition, there is an increased prevalence of cerebrovascular disease among patients with PAD.

PAD can be treated using percutaneous translumenal balloon angioplasty (PTA). The use of stents in conjunction with PTA decreases the incidence of restenosis. However, the post-operative results obtained with medical devices such as stents do not match the results obtained using standard operative revascularization procedures, i.e., those using a venous or prosthetic bypass material. (Principles of Surgery, Schwartz et al. eds., Chapter 20, Arterial Disease, 7th Edition, McGraw-Hill Health Professions Division, New York 1999).

Preferably, PAD is treated using bypass procedures where the blocked section of the artery is bypassed using a graft. (Principles of Surgery, Schwartz et al. eds., Chapter 20, Arterial Disease, 7th Edition, McGraw-Hill Health Professions Division, New York 1999). The graft can consist of an autologous venous segment such as the saphenous vein or a synthetic graft such as one made of polyester, polytetrafluoroethylene (PTFE), or expanded polytetrafluoroethylene (ePTFE). The post-operative patency rates depend on a number of different factors, including the luminal dimensions of the bypass graft, the type of synthetic material used for the graft and the site of outflow. Restenosis and thrombosis, however, remain significant problems even with the use of bypass grafts. For example, the patency of infrainguinal bypass procedures at 3 years using an ePTFE bypass graft is 54% for a femoral-popliteal bypass and only 12% for a femoral-tibial bypass.

Consequently, there is a significant need to improve the performance of both stents and synthetic bypass grafts in order to further reduce the morbidity and mortality of CAD and PAD.

With stents, the approach has been to coat the stents with various anti-thrombotic or anti-restenotic agents in order to reduce thrombosis and restenosis. For example, impregnating stents with radioactive material appears to inhibit restenosis by inhibiting migration and proliferation of myofibroblasts. (U.S. Pat. Nos. 5,059,166, 5,199,939 and 5,302,168). Irradiation of the treated vessel can pose safety problems for the physician and the patient. In addition, irradiation does not permit uniform treatment of the affected vessel.

Alternatively, stents have also been coated with chemical agents such as heparin or phosphorylcholine, both of which appear to decrease thrombosis and restenosis. Although heparin and phosphorylcholine appear to markedly reduce restenosis in animal models in the short term, treatment with these agents appears to have no long-term effect on preventing restenosis. Additionally, heparin can induce thrombocytopenia, leading to severe thromboembolic complications such as stroke. Therefore, it is not feasible to load stents with sufficient therapeutically effective quantities of either heparin or phosphorylcholine to make treatment of restenosis in this manner practical.

Synthetic grafts have been treated in a variety of ways to reduce postoperative restenosis and thrombosis. (Bos et al. 1998. Small-Diameter Vascular Graft Prostheses:Current Status Archives Physio. Biochem. 106:100-115). For example, composites of polyurethane such as meshed polycarbonate urethane have been reported to reduce restenosis as compared with ePTFE grafts. The surface of the graft has also been modified using radiofrequency glow discharge to add polyterephalate to the ePTFE graft. Synthetic grafts have also been impregnated with biomolecules such as collagen.

U.S. Pat. Nos. 5,288,711; 5,563,146; 5,516,781, and 5,646,160 disclose a method of treating hyperproliferative vascular disease with rapamycin alone or in combination with mycophenolic acid. The rapamycin is given to the patient by various methods including, orally, parenterally, intravascular, intranasally, intrabronchially, transdermally, rectally, etc. The patents further disclose that the rapamycin can be provided to the patient via a vascular stent, which is impregnated with the rapamycin alone or in combination with heparin or mycophenolic acid. One of the problems encountered with the impregnated stent of the patents is that the drug is released immediately upon contact with the tissue and does not last for the amount of time required to prevent restenosis.

European Patent Application No. EP 0 950 386 discloses a stent with local rapamycin delivery, in which the rapamycin is deliver to the tissues directly from micropores in the stent body, or the rapamycin is mixed or bound to a polymer coating applied on the stent. EP 0 950 386 further discloses that the polymer coating consists of purely nonabsorbable polymers such as polydimethylsiolxane, poly(ethylene-vingylacetate), acrylate based polymers or copolymers, etc. Since the polymers are purely nonabsorbable, after the drug is delivered to the tissues, the polymers remain at the site of implantation. Nonabsorbable polymers remaining in large amounts adjacent to the tissues have been known to induce inflammatory reactions on their own and restenosis recurs at the implantation site thereafter.

Additionally, U.S. Pat. No. 5,997,517 discloses a medical device coated with a thick coherent bond coat of acrylics, epoxies, acetals, ethylene copolymers, vinyl polymers and polymers containing reactive groups. The polymers disclosed in the patent are also nonabsorbable and may cause side effects when used in medical device for implantation similarly as discussed above with respect to EP 0 950 386.

None of the aforementioned approaches has significantly reduced the incidence of thrombosis or restenosis over an extended period of time. Additionally, the coating of prior art medical devices have been shown to crack upon implantation of the devices. Therefore, new devices and methods of treatment are needed to treat vascular disease.

### SUMMARY OF INVENTION

The invention relates to a medical device for implanting into the lumen of a blood vessel or an organ with a lumen. The medical device is, for example, a stent or a synthetic graft having a structure adapted for the introduction into a patient. The device is coated with a matrix comprising a bioabsorbable material which is a nontoxic, biocompatible, bioerodible and biodegradable synthetic material, and at least one pharmaceutical substance or composition for delivering a drug or pharmaceutical substance to the tissues adjacent to the site of implantation. The pharmaceutical substance or composition inhibits smooth muscle cell migration, and prevents restenosis after implantation of the medical device.

In one embodiment, the implantable medical device comprises a stent. The stent can be selected from uncoated stents available in the art. In accordance with one embodiment of the invention, the stent is an expandable intraluminal endoprosthesis comprising a tubular member as described in U.S. patent application Ser. No. 09/094,402, which disclosure is herein incorporated by reference in its entirety.

The matrix comprises a polymer, oligomer or co-polymer for coating the medical device from various types and sources, including, natural or synthetic polymers, which are biocompatible, biodegradable, bioabsorbable and useful for controlled-released of the medicament. For example, the synthetic material can be selected from polyesters such as polylactic acid, polyglycolic acid or copolymers thereof, a polyanhydride, polycaprolactone, polyhydroxybutyrate valerate, and other biodegradable polymer, or mixtures or copolymers. In another embodiment, the naturally occurring polymeric materials can be selected from proteins such as collagen, fibrin, elastin, and extracellular matrix component, or other biologic agents or mixtures. The polymer material can be applied together as a composition with the pharmaceutical substance on the surface of the medical device as a single layer. Multiple layers of composition can be applied as the coat. In another embodiment of the invention, multiple layers of the polymer can be applied between layer of the pharmaceutical substance. For example, the layers may be applied sequentially, with the first layer directly in contact with the stent or synthetic graft surface and the second layer comprising the pharmaceutical substance and having one surface in contact with the first layer and the opposite surface in contact with a third layer of polymer which is in contact with the surrounding tissue. Additional layers of the polymer and drug composition can be added as required, alternating each component or mixtures of components thereof.

In one embodiment of the invention, the matrix comprises poly(lactide-coglycolide) as the matrix polymer for coating the medical device. In this embodiment of the invention, the poly(lactide-co-glycolide) composition comprises at least one polymer of poly-DL-co-glycolide or a copolymer or mixtures thereof, and is mixed together with the pharmaceutical substances to be delivered to the tissues. The coating composition is then applied to the surface of the device using standard techniques, such as spraying or dipping. Alternatively, the poly(lactide-co-glycolide) solution can be applied as a single layer separating a layer or layers of the pharmaceutical substance(s).

In another embodiment of the invention, the coating composition further comprises a nonabsorbable polymer, such as ethylene vinyl acetate (EVAC) and methylmethacrylate (MMA). The nonabsorbable polymer aids in the controlled release of the substance so as to increase the molecular weight of the composition, thereby delaying or slowing the rate of release of the pharmaceutical substance.

Compounds or pharmaceutical compositions which can be incorporated in the matrix, include, but are not limited to immunosuppressant drugs, drugs which inhibit smooth muscle cell proliferation, antithrombotic drugs such as thrombin inhibitors, antiinflammatory drugs, growth factors which induce endothelial cell growth and differentiation, peptides or antibodies which inhibit mature leukocyte adhesion, antibiotics/antimicrobials, statins, and the like.

The invention also relates to a method for administering a pharmaceutical substance locally to a patient in need of such substance. The method comprises administering a coated medical device to the patient, wherein the coating comprises a pharmaceutical substance for inhibiting restenosis and a bioabsorbable, biocompatible, biodegradable, bioerodible, nontoxic polymer matrix, comprising polylactic acid polymer, polyglycolic acid polymer, copolymers of polylactic and polyglycolic acid, or mixtures thereof.

The invention also relates to a method of making the coated medical device of the invention. In one embodiment, the medical device is coated with a solution comprising a bioabsorbable, biocompatible, biodegradable, nontoxic polymer matrix, such as poly(lactide-co-glycolide) copolymer and the pharmaceutical substance. In the method, the polymer matrix and the substance are mixed prior to applying the coat on the medical device. The polymer matrix containing the pharmaceutical substance can be applied to the medical device by several methods using standard techniques.

### BRIEF DESCRIPTION OF DRAWINGS

FIG. 1 as an illustration of a coated stent with a poly(DL-Lactide-co-Glycolide)-based matrix in accordance with the invention.

FIG. 2 is a graph showing the drug elution profile of a drug-coated stent incubated for 21 days in bovine serum, wherein the coating comprised 500 μg of 4% Paclitaxel and 96% polymer. The polymer used in the coating was 50:50 Poly(DL Lactide-co-Glycolide).

FIG. 3 is a graph showing the drug elution profile of a drug-coated stent incubated for 10 days in bovine serum, wherein the coating comprised 500 μg of 8% Paclitaxel and 92% polymer. The polymer used in the coating was 50:50 Poly-DL lactide/EVAC 25.

FIG. 4 is a graph showing the drug elution profile of a drug-coated stent incubated for 14 days in bovine serum, wherein the coating comprised 500 μg of 8% Paclitaxel and 92% polymer. The polymer used in the coating was 80:20 Poly-DL Lactide/EVAC 25.

FIG. 5 is a graph showing the drug elution profile of a drug-coated stent incubated for 21 days in bovine serum, wherein the coating comprised 500 μg of 8% Paclitaxel and 92% poly(DL-lactide) polymer.

### DETAILED DESCRIPTION

The invention is directed to a medical device in the form of an implantable structure, which is coated with a homogenous matrix comprising a pharmaceutical substance and a biodegradable, biocompatible, non-toxic, bioerodible, bioabsorbable polymer matrix. The structure of the device has at least one surface and comprises at least one or more based materials. The based materials can be selected from stainless steel, Nitinol, MP35N, gold, tantalum, platinum or platinum irdium, or other biocompatible metals and/or alloys such as carbon or carbon fiber, cellulose acetate, cellulose nitrate, silicone, cross-linked polyvinyl alcohol (PVA) hydrogel, cross-linked PVA hydrogel foam, polyurethane, polyamide, styrene isobutylene-styrene block copolymer (Kraton), polyethylene teraphthalate, polyurethane, polyamide, polyester, polyorthoester, polyanhidride, polyether sulfone, polycarbonate, polypropylene, high molecular weight polyethylene, polytetrafluoroethylene, or other biocompatible polymeric material, or mixture of copolymers thereof; polyesters such as, polylactic acid, polyglycolic acid or copolymers thereof, a polyanhydride, polycaprolactone, polyhydroxybutyrate valerate or other biodegradable polymer, or mixtures or copolymers, extracellular matrix components, proteins, collagen, fibrin or other bioactive agent, or mixtures thereof.

The medical device of the invention can be any device that is introduced temporarily or permanently into a mammal for the prophylaxis or therapy of a medical condition. These devices include any that are introduced subcutaneously, percutaneously or surgically to rest within an organ, tissue or lumen of an organ, such as arteries, veins, ventricles or atrium of the heart. Medical devices may include stents, stent grafts; covered stents such as those covered with polytetrafluoroethylene (PTFE), expanded polytetrafluoroethylene (ePTFE), or synthetic vascular grafts, artificial heart valves, artificial hearts and fixtures to connect the prosthetic organ to the vascular circulation, venous valves, abdominal aortic aneurysm (AAA) grafts, inferior venal caval filters, permanent drug infusion catheters, embolic coils, embolic materials used in vascular embolization (e.g., cross-linked PVA hydrogel), vascular sutures, vascular anastomosis fixtures, transmyocardial revascularization stents and/or other conduits.

The coating composition on the medical device comprises one or more pharmaceutical substances incorporated into a polymer matrix so that the pharmaceutical substance(s) is released locally into the adjacent or surrounding tissue in a slow or controlled-release manner. The release of the pharmaceutical substance in a controlled manner allows for smaller amounts of drug or active agent to be released for a long period of time in a zero order elution profile manner. The release kinetics of a drug further depends on the hydrophobicity of the drug, i.e., the more hydrophobic the drug is, the slower the rate of release of the drug from the matrix. Alternative, hydrophilic drugs are released from the matrix at a faster rate. Therefore, the matrix composition can be altered according to the drug to be delivered in order to maintain the concentration of drug required at the site for a longer period of time. The invention, therefore, provides a long term effect of the drugs at the required site which is more efficient in preventing restenosis and minimizes the side effects of the released pharmaceutical substances used.

The matrix can be selected from a variety of polymer matrices. However, the matrix should be biocompatible, biodegradable, bioerodible, non-toxic, bioabsorbable, and with a slow rate of degradation. Biocompatible matrices that can be used in the invention include, but are not limited to, poly(lactide-co-glycolide), polyesters such as polylactic acid, polyglycolic acid or copolymers thereof, polyanhydride, polycaprolactone, polyhydroxybutyrate valerate, and other biodegradable polymer, or mixtures or copolymers, and the like. In another embodiment, the naturally occurring polymeric materials can be selected from proteins such as collagen, fibrin, elastin, and extracellular matrix components, or other biologic agents or mixtures thereof.

Polymer matrices used with the coating of the invention such as poly(lactide-co-glycolide); poly-DL-lactide, poly-L-lactide, and/or mixtures thereof are of various inherent viscosities and molecular weights. For example, in one embodiment of the invention, poly(DL lactide-co-glycolide) (DLPLG, Birmingham Polymers Inc.) is used. Poly(DL-lactide-co-glycolide) is a bioabsorbable, biocompatible, biodegradable, non-toxic, bioerodible material, which is a vinylic monomer and serves as a polymeric colloidal drug carrier. The poly-DL-lactide material is in the form of homogeneous composition and when solubilized and dried, it forms a lattice of channels in which pharmaceutical substances can be trapped for delivery to the tissues.

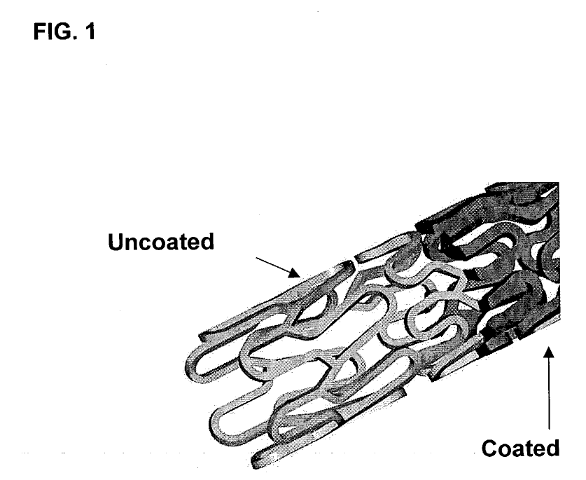

The drug release kinetics of the coating on the device of the invention can be controlled depending on the inherent viscosity of the polymer or copolymer used as the matrix and the amount of drug in the composition. The polymer or copolymer characteristics can vary depending on the inherent viscosity of the polymer or copolymer. For example, in one embodiment of the invention using poly(DL-lactide-co-glycolide), the inherent viscosity can range from about 0.55 to 0.75 (dL/g). Poly(DL-Lactide-co-Glycolide) can be added to the coating composition from about 50 to about 99% (w/w) of the polymeric composition. FIG. 1 shows a stent partially coated with the coating comprising poly(DL-lactide-co-glycolide) polymer matrix. The poly(DL-lactide-co-glycolide) polymer coating deforms without cracking, for example, when the coated medical device is subjected to stretch and/or elongation and undergoes plastic and/or elastic deformation. Therefore, polymers which can withstand plastic and elastic deformation such as poly(DL-lactide-co-glycolide) acid-based coats, have advantageous characteristics over prior art polymers. The rate of dissolution of the matrix can also be controlled by using polymers of various molecular weight. For example, for slower rate of release of the pharmaceutical substances, the polymer should be of higher molecular weight. By varying the molecular weight of the polymer or combinations thereof, a preferred rate of dissolution can be achieved for a specific drug. Alternatively, the rate of release of pharmaceutical substances can be controlled by applying a polymer layer to the medical device, followed by one or more than one layer of drugs, followed by one or more layers of the polymer. Additionally, polymer layers can be applied between drug layers to decrease the rate of release of the pharmaceutical substance from the coating.

The malleability of the coating composition of the invention can be further improved by varying the ratio of lactide to glycolide in the copolymer. That is, the ratio of components of the polymer can be adjusted to make the coating more malleable and to enhance the mechanical adherence of the coating to the surface of the medical device and aid in the release kinetics of the coating composition. In this embodiment of the invention, the polymer can vary in molecular weight depending on the rate of drug release desired. The ratio of lactide to glycolide can range, respectively, from about 50-85% to 50-15% in the composition. By adjusting the amount of lactide in the polymer, the rate of release of the drugs from the coating can also be controlled.

The characteristic biodegradation of the polymer, therefore, to some degree determines the rate of drug release from the coating. Information on the biodegradation of polymers can be obtained from the manufacturer information, for example, from Birmingham Polymers.

The principle mode of degradation for the lactide and glycolide polymers and copolymers is hydrolysis. Degradation proceeds first by diffusion of water into the material followed by random hydrolysis, fragmentation of the material and finally a more extensive hydrolysis accompanied by phagocytosis, diffusion and metabolism. The hydrolysis is affected by the size and hydrophillicity of the particular polymer, the crystallinity of the polymer and the pH and temperature of the environment.

In general, the degradation time will be shorter for low molecular weight polymers, more hydrophillic polymers, more amorphous polymers and copolymers higher in glycolide. Therefore at identical conditions, low molecular weight copolymers of DL Lactide and Glycolide, such as 50/50 DL-PLG will degrade relatively rapidly whereas the higher molecular weight homopolymers such as L-PLA will degrade much more slowly.

Once the polymer is hydrolyzed, the products of hydrolysis are either metabolized or secreted. The lactic acid generated by the hydrolytic degradation of PLA becomes incorporated into the tricarboxylic acid cycle and is secreted as carbon dioxide and water. PGA is also broken down by random hydrolysis accompanied by non-specific enzymatic hydrolysis to glycolic acid which is either secreted or enzymatically converted to other metabolized species.

In another embodiment, the coating composition comprises a nonabsorbable polymer, such as ethylene vinyl acetate (EVAC), poly butyl methacrylate (PBMA) and methylmethacrylate (MMA) in amounts from about 0.5 to about 99% of the final composition. The addition of EVAC, PBMA or methylmethacrylate increases malleability of the matrix so that the device is more plastically deformable. The addition of methylmethacrylate to the coating delays the degradation of the coat and therefore, improves the controlled release of the coat, so that the pharmaceutical substance is released at a slower rate.

The coating of the medical device can be applied to the medical device using standard techniques to cover the entire surface of the device or partially, as a single layer of a homogeneous mixture of drugs and matrix, and is applied in a thickness of from about 1 to 100 mm. Alternative, multiple layers of the matrix/drug composition can be applied on the surface of the device. For example, multiple layers of various pharmaceutical substances can be deposited onto the surface of the medical device so that a particular drug can be released at one time, one drug in each layer, which can be separated by polymer matrix. The active ingredient or pharmaceutical substance component of the composition can range from about 1 to about 60% (w/w) or the composition. Upon contact of the coating composition with adjacent tissue where implanted, the coating begins to degrade in a controlled manner. As the coating degrades, the drug is slowly released into adjacent tissue and the drug is eluted from the device, thereby, preventing restenosis. Additionally, since the polymers of the invention form a lattice of channels, the drugs are slowly released from the channels upon implantation of the device. Therefore, the present invention provides an improved mechanism of delivering a drug to surrounding tissue from a coated medical device. That is, drug elution via channels in the coating matrix and degradation of the matrix. The coating of the invention can be made so that the drug provided can elute from the surface of the medical device for a period from the implant to about a year. The drug may elute by erosion as well as diffusion when drug concentrations are low. With high concentrations of drug, the drug may elute via channels in the coating matrix.

The pharmaceutical substance of the invention includes drugs which are used in the treatment of restenosis. For example, the pharmaceutical substances include, but are not limited to antibiotics/antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, peptides, proteins, extracellular matrix components, vasodialators, thrombolytics, anti-metabolites, growth factor agonists, antimitotics, steroidal and nonsterodial antiinflammatory agents, angiotensin converting enzyme (ACE) inhibitors, free radical scavengers, anti-cancer chemotherapeutic agents. For example, some of the aforementioned pharmaceutical substances include, cyclosporins A (CSA), rapamycin, mycophenolic acid (MPA), retinoic acid, vitamin E, probucol, L-arginine-L-glutamate, everolimus, and paclitaxel.

The invention also relates to a method of treating a patient having vascular disease and in need of such treatment with the coated medical device of the invention. The method comprises administering to the patient a coated medical device of the invention.

The following examples illustrate the invention, but in no way limit the scope of the invention.

### EXAMPLE 1

Preparation of Coating Composition

The polymer Poly DL Lactide-co-Glycolide (DLPLG, Birmingham Polymers) is provided as a pellet. To prepare the polymer matrix composition for coating a stent, the pellets are weighed and dissolved in a ketone or methylene chloride solvent to form a solution. The drug is dissolved in the same solvent and added to the polymer solution to the required concentration, thus forming a homogeneous coating solution. To improve the malleability and change the release kinetics of the coating matrix, the ratio of lactide to glycolide is varied. This solution is then used to coat the stent to form a uniform coating as shown in FIG. 1. Alternatively, the polymer(s)/drug(s) composition can be deposited on the surface of the stent using standard methods.

### EXAMPLE 2

Evaluation of Polymer/Drugs and Concentrations

Process for Spray-Coating Stents

The polymer pellets of DLPLG which have been dissolved in a solvent are mixed with one or more drugs. Alternatively, one or more polymers can be dissolved with a solvent and one or more drugs can be added and mixed. The resultant mixture is applied to the stent uniformly using standard methods. After coating and drying, the stents are evaluated. The following list illustrates various examples of coating combinations, which were studied using various drugs and comprising DLPLG and/or combinations thereof. In addition, the formulation can consist of a base coat of DLPLG and a top coat of DLPLG or another polymer such as DLPLA or EVAC 25. The abbreviations of the drugs and polymers used in the coatings are as follows: MPA is mycophenolic acid, RA is retinoic acid; CSA is cyclosporine A; LOV is lovastatin™ (mevinolin); PCT is Paclitaxel; PBMA is Poly butyl methacrylate, EVAC is ethylene vinyl acetate copolymer; DLPLA is Poly (DL Lactide), DLPLG is Poly(DL Lactide-co-Glycolide).

Examples of the coating components and amounts (%) which can be used in the invention comprise:

1. 50% MPA/50% Poly L Lactide

2. 50% MPA/50% Poly DL Lactide

3. 50% MPA/50% (86:14 Poly DL Lactide-co-Caprolactone)

4. 50% MPA/50% (85:15 Poly DL Lactide-co-Glycolide)

5. 16% PCT/84% Poly DL Lacide

6. 8% PCT/92% Poly DL Lactide

7. 4% PCT/92% Poly DL Lactide

8. 2% PCT/92% Poly DL Lactide

9. 8% PCT/92% of (80:20 Poly DL Lactide/EVAC 40)

10. 8% PCT/92% of (80:20 Poly DL Lactide/EVAC 25)

11. 4% PCT/96% of (50:50 Poly DL Lactide/EVAC 25)

12. 8% PCT/92% of (85:15 Poly DL Lactide-co-Glycolide)

13. 4% PCT/96% of (50:50 Poly DL Lactide-co-Glycolide)

14. 25% LOV/25% MPA/50% of (EVAC 40/PBMA)

15. 50% MPA/50% of (EVAC 40/PBMA)

16. 8% PCT/92% of (EVAC 40/PBMA)

17. 8% PCT/92% EVAC 40

18. 8% PCT/92% EVAC 12

19. 16% PCT/84% PBMA

20. 50% CSA/50% PBMA

21. 32% CSA/68% PBMA

22. 16% CSA/84% PBMA

### EXAMPLE 3

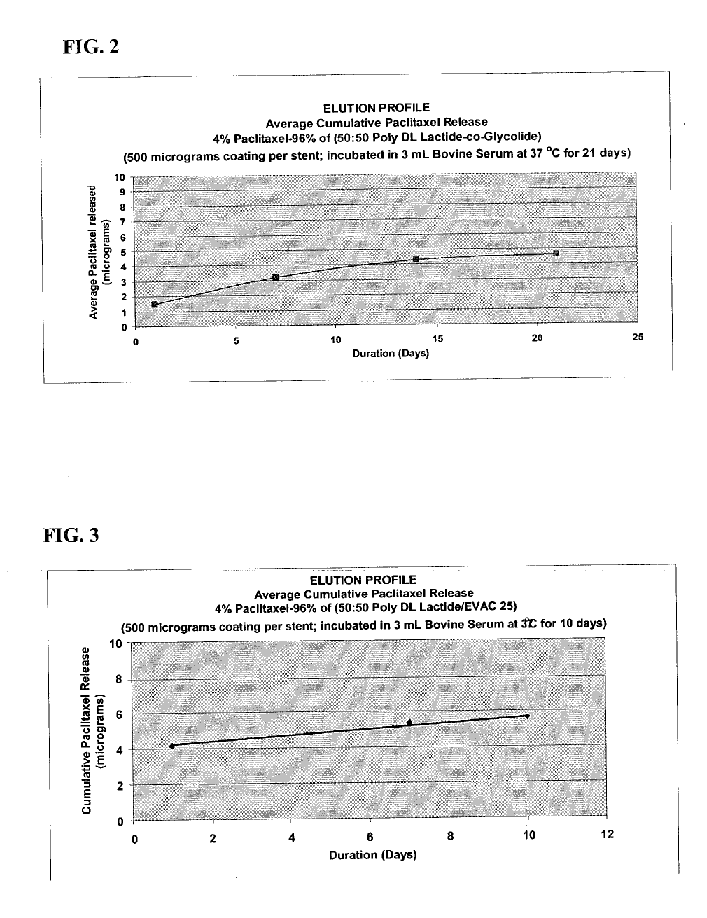

The following experiments were conducted to measure the drug elution profile of the coating on stents coated by the method described in Example 2. The coating on the stent consisted of 4% Paclitaxel and 96% of a 50:50 Poly(DL-Lactide-co-Glycolide) polymer. Each stent was coated with 500 μg of coating composition and incubated in 3 ml of bovine serum at 37° C. for 21 days. Paclitaxel released into the serum was measured using standard techniques at various days during the incubation period. The results of the experiments are shown in FIG. 2. As shown in FIG. 2, the elution profile of Paclitaxel release is very slow and controlled since only about 4 μg of Paclitaxel are released from the stent in the 21-day period.

### EXAMPLE 4

The following experiments were conducted to measure the drug elution profile of the coating on stents coated by the method describe in Example 2. The coating on the stent consisted of 4% Paclitaxel and 92% of a 50:50 of Poly(DL-Lactide) and EVAC 25 polymer. Each stent was coated with 500 μg of coating composition and incubated in 3 ml of bovine serum at 37° C. for 10 days. Paclitaxel released into the serum was measured using standard techniques at various days during the incubation period. The results of the experiments are shown in FIG. 3. As shown in FIG. 3, the elution profile of Paclitaxel release is very slow and controlled since only about 6 μg of Paclitaxel are released from the stent in the 10-day period.

### EXAMPLE 5

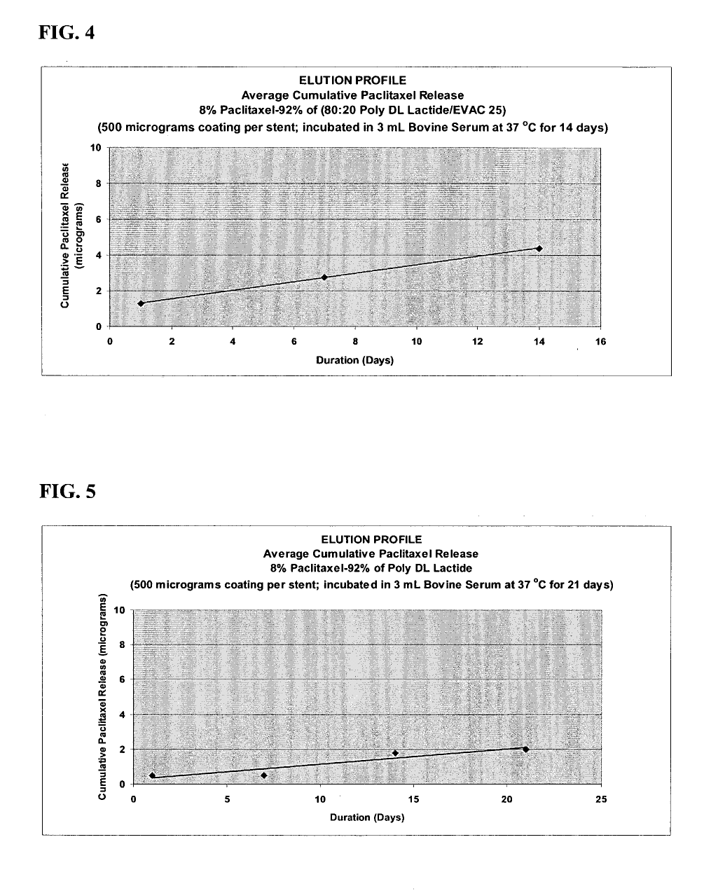

The following experiments were conducted to measure the drug elution profile of the coating on stents coated by the method describe in Example 2. The coating on the stent consisted of 8% Paclitaxel and 92% of a 80:20 of Poly(DL-Lactide) and EVAC 25 polymer. Each stent was coated with 500 μg of coating composition and incubated in 3 ml of bovine serum at 37° C. for 14 days. Paclitaxel released into the serum was measured using standard techniques at various days during the incubation period. The results of the experiments are shown in FIG. 4. As shown in FIG. 4, the elution profile of Paclitaxel release is very slow and controlled since only about 4 μg of Paclitaxel are released from the stent in the 14-day period.

### EXAMPLE 6

The following experiments were conducted to measure the drug elution profile of the coating on stents coated by the method describe in Example 2. The coating on the stent consisted of 8% Paclitaxel and 92% of Poly(DL-Lactide) polymer. Each stent was coated with 500 μg of coating composition and incubated in 3 ml of bovine serum at 37° C. for 21 days. Paclitaxel released into the serum was measured using standard techniques at various days during the incubation period. The results of the experiments are shown in FIG. 5. As shown in FIG. 5, the elution profile of Paclitaxel release is very slow and controlled since only about 2 μg of Paclitaxel are released from the stent in the 21-day period.

The above data show that by varying the polymer components of the coating, the release of a drug can be controlled for a period of time required.

What is claimed is:

1. A medical device comprising a coating for controlled release of one or more pharmaceutical substances to adjacent tissue for inhibiting restenosis, wherein the coating comprises a bio-absorbable matrix and one or more pharmaceutical substances.

2. The medical device of claim 1, wherein the device is structured and configured to be implanted in a patient, and wherein at least one surface of the device comprises one or more based materials.

3. The medical device of claim 1, wherein the medical device is a stent, a vascular or other synthetic graft, or a stent in combination with a synthetic graft.

4. The medical device of claim 1, wherein the medical device is a vascular stent.

5. The medical device of claim 2, wherein the based material is biocompatible.

6. The medical device of claim 1, wherein the based material is selected from group consisting of stainless steel, Nitinol, MP35N, gold, tantalum, platinum or platinum irdium, biocompatible metals and/or alloys, carbon fiber, cellulose acetate, cellulose nitrate, silicone, cross-linked polyvinyl acetate (PVA) hydrogel, cross-linked PVA hydrogel foam, polyurethane, polyamide, styrene isobutylene-styrene block copolymer (Kraton), polyethylene teraphthalate, polyurethane, polyamide, polyester, polyorthoester, polyanhidride, polyether sulfone, polycarbonate, polypropylene, high molecular weight polyethylene, polytetrafluoroethylene, polyesters of polylactic acid, polyglycolic acid, copolymers thereof, a polyanhydride, polycaprolactone, polyhydroxybutyrate valerate, extracellular matrix components, proteins, collagen, fibrin, and mixtures thereof.

7. The medical device of claim 1, wherein the bioabsorbable matrix comprises one or more polymers or oligomers and is selected from the group consisting of poly(lactide-co-glycolide), polylactic acid, polyglycolic acid, a polyanhydride, polycaprolactone, polyhydroxybutyrate valerate, and mixtures or copolymers thereof.

8. The medical device of claim 1, wherein the coating comprises poly(DL-lactide-co-glycolide).

9. The medical device of claim 7, wherein the bio-absorbable matrix comprises poly(DL-lactide).

10. The medical device of claim 1, wherein the pharmaceutical substance is selected from the group consisting of antibiotics/antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, peptides, proteins, extracellular matrix components, vasodialators, thrombolytics, anti-metabolites, growth factor agonists, antimitotics, steroidal and nonsterodial antiinflammatory agents, angiotensin converting enzyme(ACE) inhibitors, free radical scavangers, and anti-cancer chemotherapeutic agents.

11. The medical device of claim 10, wherein the pharmaceutical substance is selected from the group consisting of paclitaxel, cyclosporin A, mycophenolic acid, mycophenolate mofetil acid, rapamycin, azathioprene, tacrolimus, tranilast, dexamethasone, other corticosteroid, everolimus, retinoic acid, vitamin E, statins, and probucol.

12. The medical device of claim 11, wherein the pharmaceutical substances are cyclosporin A and mycophenolic acid.

13. The medical device of claim 11, wherein the pharmaceutical substances are mycophenolic acid and vitamin E.

14. The medical device of claim 8, wherein the poly(D,L-lactide-co-glycolide) comprises from about 50 to 99% of the polymer in the coating.

15. The medical device of claim 8, wherein the poly(DL-lactide-co-glycolide) polymer comprises from about 50 to 85% lactide polymer and from about 15 to 50% glycolide polymer.

16. The medical device of claim 1, wherein the pharmaceutical substance comprises from about 1 to about 50% (w/w) of the composition.

17. The medical device of claim 12, wherein the pharmaceutical substance is paclitaxel and/or cyclosporin A.

18. The medical device of claim 1 or 2, further comprising a nonabsorbable polymer.

19. The medical device of claim 18, wherein the nonabsorbable polymer is ethylene vinyl acetate or methylmethacylate.

20. The medical device of claim 19, wherein the ethylene vinyl acetate is ethylene vinyl acetate 25.

21. The medical device of claim 1, wherein the coating comprises a single homogeneous layer comprising poly(DL-lactide-co-glycolide) and the pharmaceutical substances.

22. The medical device of claim 1, wherein the coating comprises multiple layers of the poly(DL-lactide-co-glycolide) polymer and the pharmaceutical substance.

23. The medical device of claim 1, wherein the coating comprises multiple layers of the pharmaceutical substances and multiple layers of poly(DL-lactide-co-glycolide) polymer.

24. A method for preparing a coated medical device according to claims 1-23 comprising the steps of:
applying to a surface of the medical device a coating composition comprising one or more bioabsorbable polymers and one or more pharmaceutical substance, and
drying the coating on the device.

25. The method of claim 24, wherein the composition further comprises one or more nonabsorbable polymer.

26. A method of treating vascular disease, comprising implanting the medical device of claims 1-23 into a patient in need of such treatment.
